(19) United States
(12) Patent Application Publication
(10) Pub. No.: US 2021/0280305 A1
(43) Pub. Date: Sep. 9, 2021

(54) DATA LOGGER FOR STERILIZER

(71) Applicant: Midmark Corporation, Versailles, OH (US)

(72) Inventors: Gary W. Benning, Greenville, OH (US); David P. Hosey, Minster, OH (US); Steffen Baumann, Dayton, OH (US); Zachary Edward Baltrip, Troy, OH (US); Douglas R. Hall, Troy, OH (US)

(21) Appl. No.: 17/195,906

(22) Filed: Mar. 9, 2021

Related U.S. Application Data
(60) Provisional application No. 62/987,087, filed on Mar. 9, 2020.

(51) Int. Cl. G16H 40/20; G06F 16/25; G06F 13/16

(52) U.S. Cl. CPC G16H 40/20; G06F 13/1668; G06F 16/258

(57) ABSTRACT

A data logger for collecting, storing, and transferring data received from a sterilizer is described herein. The data logger is sized and shaped to fit into a slot or opening defined by the sterilizer and previously used for receiving a printing device to output sterilizer data on a printer ribbon. The data logger includes a circuit to facilitate receiving data formatted to conform to at least two different transmission protocols (e.g. RS-232 serial data transmission protocol or TTL serial data transmission protocol). The circuit allows the data logger to be used with different styles of sterilizers and thus increases the set of viable sterilizers. The data logger includes at least one memory device for storing the logged data. This memory device may be a removable component. The data logger may also include a port or connector element for a second memory device such as a USB stick. The data logger may be configured to transfer the logged data to the USB stuck upon insertion or detection of the USB stick in the port.

### BACKGROUND

Sterilizers or autoclaves are used for sterilizing medical instruments to prepare the instruments for subsequent sterile use in a medical procedure. In general, these box-like devices define an internal cavity with a selectively openable door. Once instruments are disposed within the cavity, the user actuates a control panel to begin the sterilizing process. The device applies heat, steam, and/or pressure to the medical instruments for a set amount of time to process and sterilize the instruments therein.

Sterilization data such as the cycle number, time, date, timestamped temperature achieved within the chamber, timestamped pressure within the chamber, drying time, and overall processing time may be captured by the sterilizer. Data relating to errors in the sterilizing process may also be captured and used to determine whether instruments were properly sterilized and the cause for the error.

Heretofore, sterilizer data was output via a printer attached or disposed within the sterilizer. However, sterilizer printers produce noise, which may disturb others within range of the sterilizer, and also require continuous purchases of paper and ink ribbons. Sterilizer printers have been known to cause downtime attributable to loading the paper/ink as well as generally running out of these products. Further, the physical output from the printers may be lost or damaged, thus jeopardizing the collected data.

Different sterilizers may transmit the collected data using different data transmission protocols, such as the R232 serial protocol and the TTL serial protocol. Thus, a data receiving element for a particular sterilizer may not communicate with a different sterilizer. This prevents one data receiving element from being usable across multiple make/model of sterilizers.

Thus, a need exists for a device that captures data from a sterilizer, which is configured to communicate via multiple data transmission protocols, and also addresses the issues discussed above with respect to printing sterilizer data.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1 depicts a perspective view of an exemplary data logger disposed in an exemplary sterilizer;

FIG. 2 depicts a diagrammatic view of an exemplary operating environment of the data logger and sterilizer of FIG. 1;

FIG. 3 depicts a diagrammatic view of an exemplary computer system which may be used in the data logger or the sterilizer of FIG. 1;

FIG. 4 depicts a front top perspective view of the data logger of FIG. 1;

FIG. 5 depicts a rear bottom perspective view of the data logger of FIG. 1;

FIG. 6 depicts a schematic view of an exemplary internal hardware structure of the data logger of FIG. 1 including an exemplary serial data input circuit;

FIG. 7 depicts a schematic view of the serial data input circuit of FIG. 6;

FIG. 8 depicts a flowchart of an exemplary method of generating one or more records by the data logger of FIG. 1 based on events that occur during a sterilization process;

FIG. 9 depicts a flowchart of an exemplary method of updating a firmware of the data logger of FIG. 1;

FIG. 10 depicts a flowchart of an exemplary method of creating and transferring sterilization logs within the data logger of FIG. 1;

FIG. 11 depicts a flowchart of an exemplary method of creating and storing of configuration data within the data logger of FIG. 1;

FIG. 12A depicts a flowchart of a first portion of an exemplary method of projecting a menu and receiving input from a user of the data logger of FIG. 1; and

FIG. 12B depicts a flowchart of a second portion of the exemplary method of FIG. 12A.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

It will be appreciated that any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

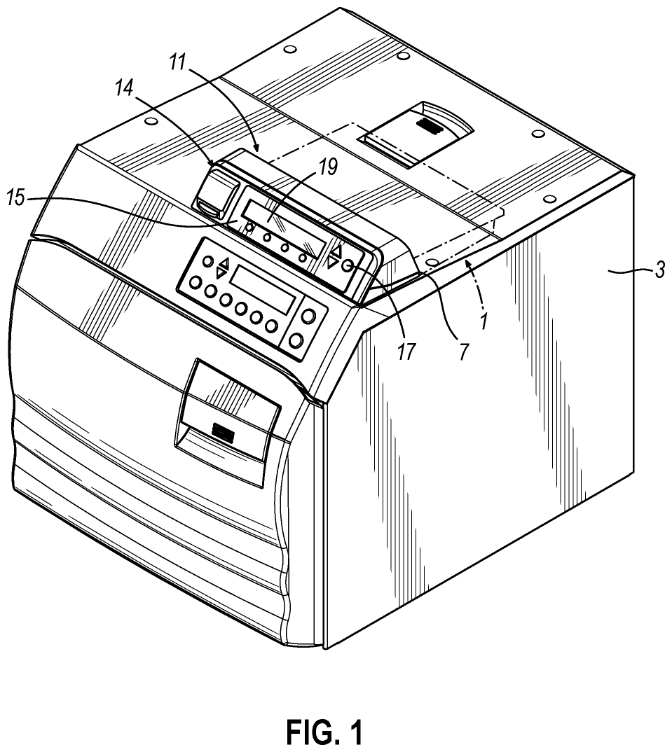

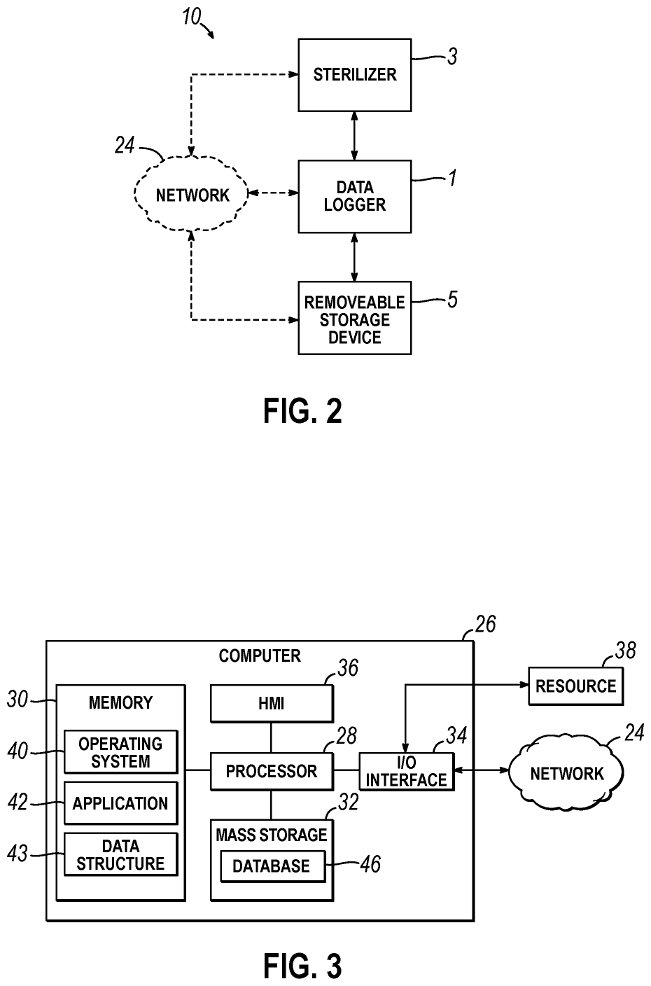

As shown in FIGS. 1 and 2, the present disclosure relates to a data logger (1) configured to be coupled with a sterilizer (3) and used to log and display sterilization information that comes from the attached sterilizer (3). Some version of data logger (1) are configured to receive +5V and serial data from sterilizer (3) over two connectors. In some version of data logger (1), data is logged to an internal microSD card and also to an external removable storage device, such as a universal serial bus (USB) memory stick if one is attached. In some version of data logger (1), the user interface consists of several membrane switch buttons and a two-line, 20-characters/line backlit LCD display. In some version of data logger (1), data logger (1) is configured to fit into the physical space on sterilizer (3) originally intended for a printer.

Some versions of data logger (1) are configured to be coupled with a removable storage device (5) for storing or otherwise providing the logged data to a user in a physically transferrable format. As shown in FIG. 2, an exemplary operating environment (10) may include data logger (1), sterilizer (3), and removable storage device (5).

### I. EXEMPLARY OPERATING ENVIRONMENT

In some versions of operating environment (10), data logger (1), sterilizer (3), and removable storage device (5) may send and receive communications between one another directly, as shown in FIG. 2. The direct communication may be through a serial data connection. Sterilizer (3) and data logger (1) may communicate using a TTL protocol for serial communication, a RS-232 protocol for serial communication, or data logger (1) may be configured with a selector circuit to enable data logger (1) to communicate using either a TTL protocol or a RS-232 protocol for serial communication, as needed by the underlying configuration of sterilizer (3).

Alternatively, in other versions of operating environment (10), data logger (1), sterilizer (3), and removable storage device (5) may communicate with each other through a network (24), as shown in FIG. 2. Network (24) may include one or more private or public networks (e.g. the Internet) that enable the exchange of data.

Referring now to FIG. 2, data logger (1), sterilizer (3), removable storage device (5), and network (24) of operating environment (10) may be implemented on one or more computing devices or systems, such as an exemplary computer system (26). Computer system (26) may include a processor (28), a memory (30), a mass storage memory device (32), an input/output (I/O) interface (34), and a Human Machine Interface (HMI) (36). Computer system (26) may also be operatively coupled to one or more external resources (38) via network (24) or I/O interface (34). External resources may include, but are not limited to, servers, databases, mass storage devices, peripheral devices, cloud-based network services, or any other suitable computer resource that may used by computer system (26).

Processor (28) may include one or more devices selected from microprocessors, micro-controllers, digital signal processors, microcomputers, central processing units, field programmable gate arrays, programmable logic devices, state machines, logic circuits, analog circuits, digital circuits, or any other devices that manipulate signals (analog or digital) based on operational instructions that are stored in memory (30). Memory (30) may include a single memory device or a plurality of memory devices including, but not limited, to read-only memory (ROM), random access memory (RAM), volatile memory, non-volatile memory, static random access memory (SRAM), dynamic random access memory (DRAM), flash memory, cache memory, or any other device capable of storing information.

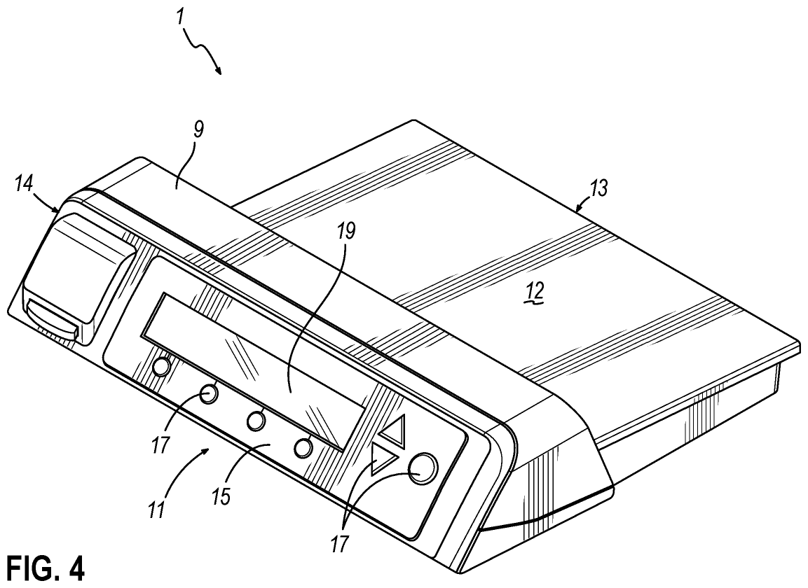

Mass storage memory device (32) may include data storage devices such as a hard drive, optical drive, tape drive, non-volatile solid-state device, or any other device capable of storing information. Further, removable storage device (5) may be represented in FIG. 3 by mass storage memory device (32), with data logger (1) being represented by computer system (26).

Processor (28) may operate under the control of an operating system (40) that resides in memory (30). Operating system (40) may manage computer resources so that computer program code embodied as one or more computer software applications, such as an application (42) residing in memory (30), may have instructions executed by processor (28). In an alternative embodiment, processor (28) may execute the application (42) directly, in which case operating system (40) may be omitted. One or more data structures (44) may also reside in memory (30), and may be used by processor (28), operating system (40), or application (42) to store or manipulate data.

I/O interface (34) may provide a machine interface that operatively couples processor (28) to other devices and systems, such as network (24) or external resource (38). Application (42) may thereby work cooperatively with network (24) or external resource (38) by communicating via I/O interface (34) to provide the various features, functions, applications, processes, or modules comprising embodiments of the invention. Application (42) may also have program code that is executed by one or more external resources (38), or otherwise rely on functions or signals provided by other system or network components external to computer system (26). Indeed, given the nearly endless hardware and software configurations possible, persons having ordinary skill in the art will understand that embodiments of the invention may include applications that are located externally to computer system (26), distributed among multiple computers or other external resources (38), or provided by computing resources (hardware and software) that are provided as a service over network (24), such as a cloud computing service.

HMI (36) may be operatively coupled to processor (28) of computer system (26) in a known manner to allow a user to interact directly with computer system (26). HMI (36) may include video or alphanumeric displays, a touch screen, a speaker, and any other suitable audio and visual indicators capable of providing data to the user. HMI (36) may also include input devices and controls such as an alphanumeric keyboard, a pointing device, keypads, pushbuttons, control knobs, microphones, etc., capable of accepting commands or input from the user and transmitting the entered input to processor (28).

A database (46) may reside on mass storage memory device (32) and may be used to collect and organize data used by the various systems and modules described herein. Database (46) may include data and supporting data structures that store and organize the data. In particular, database (46) may be arranged with any database organization or structure including, but not limited to, a relational database, a hierarchical database, a network database, or combinations thereof. A database management system in the form of a computer software application executing as instructions on processor (28) may be used to access the information or data stored in records of database (46) in response to a query, where a query may be dynamically determined and executed by operating system (40), other applications (42), or one or more modules.

### II. EXEMPLARY EXTERIOR FEATURES AND HARDWARE OF DATA LOGGER

Data logger (1) is configured to fit into the physical space on sterilizer (3) originally intended for a printer and to log and display sterilization information that is received from sterilizer (3).

A. Exemplary External Features

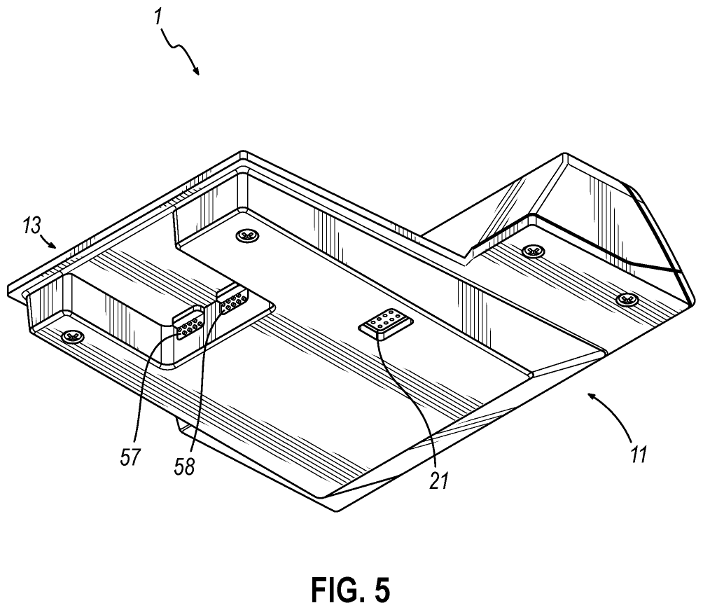

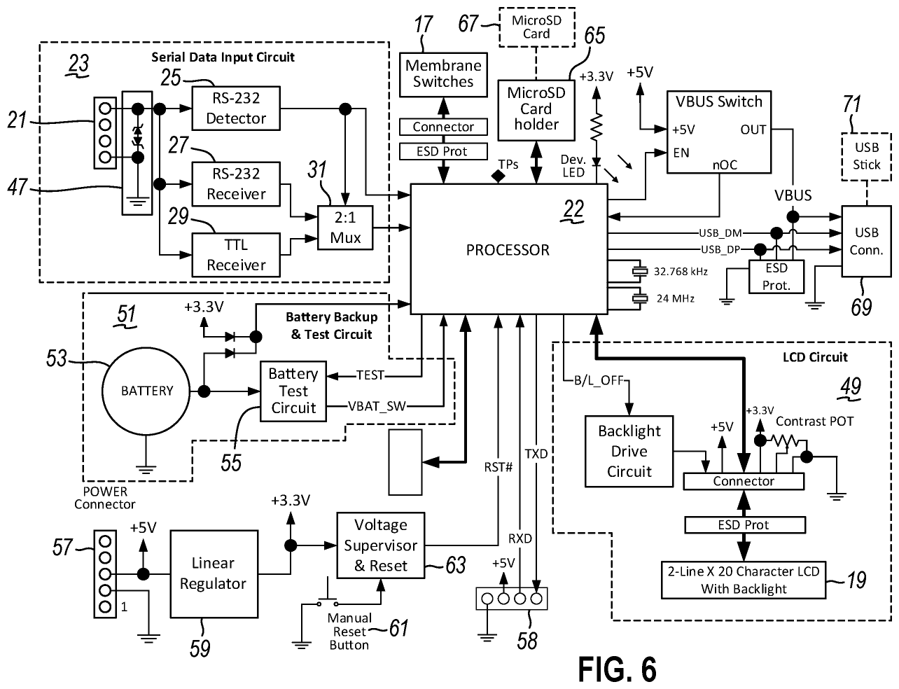

As shown in FIGS. 1, 4, and 5, sterilizer (3) defines a recess (7) sized to receive a printer device therein. Data logger (1) may be formed in a similar shape to the printer device and therefore also fit inside recess (7). Data logger (1) includes a housing (9) extending from a front side (11) to a rear side (13). Housing (9) further includes a base (12) generally disposed at rear side (13) and a user interface area (14) generally disposed at front side (11). Base (12) is complementarily sized to fit into recess (7) which in turn projects user interface area (14) toward the user of sterilizer (3). A control panel (15) is disposed within user interface area (14). Control panel (15) includes a plurality of interface buttons (17) and a display screen (19) for displaying feedback from data logger (1) to the user. In some versions of data logger (1), interface buttons (17) comprise membrane switch buttons. In some versions of data logger (1), display screen (19) comprise a backlit LCD display. As shown in FIG. 5, one or more data connector sockets (21) may be exposed through housing (9) and available for connecting with a data connection cable (not shown).

While data logger (1) is shown and described as fitting into recess (7), in other versions, data logger (1) is differently shaped and may rest beside or on top of sterilizer (3). Further, in some versions of data logger (1) may be in a different location from sterilizer (3) and communicate with sterilizer (3) (or many sterilizers) via a wireless connection over network (24).

B. Exemplary Hardware

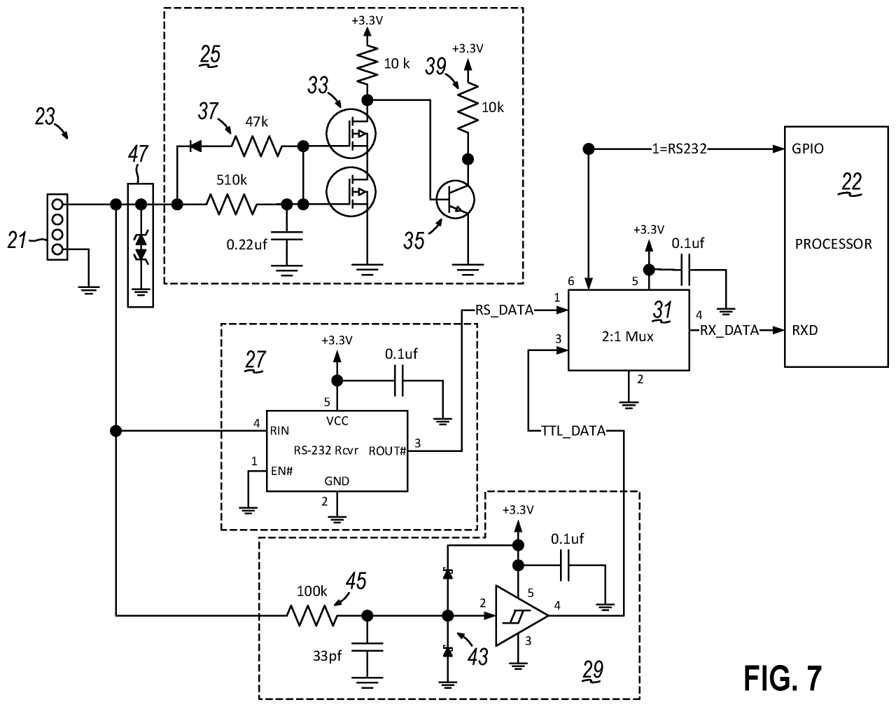

In addition to those features described above with respect to computer system (26), which may be incorporated into data logger (1), data logger further includes those features shown in FIG. 6, generally depicting a block diagram of data logger (1). An embodiment of processor (28) is shown as a processor (22). Some versions of processor (22) may comprise a microprocessor. Some versions of processor (22) may comprise an MK22FX512AVLK12 microprocessor.

i. Exemplary Serial Data Input Circuit

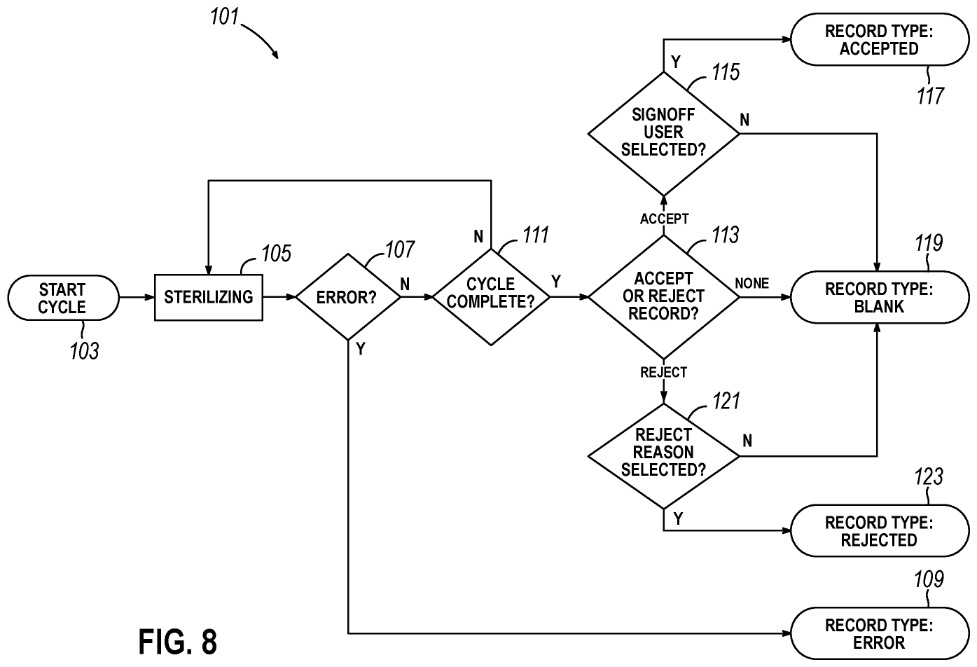

As shown generally in FIG. 6, data logger (1) includes a serial data input circuit (23), with a more specific depiction of serial data input circuit (23) shown in FIG. 7. Serial data input circuit (23) is comprised of four sub-circuits, a RS-232 detector (25), an RS-232 receiver (27), a TTL receiver (29), and a multiplexer (31), depicted as a 2:1 mux. Serial data input circuit (23) is configured to selectively receive RS-232 protocol serial data or TTL protocol serial data from sterilizer (3) and transfer either data protocol into a standardized data stream and in turn pass this data stream to processor (22) for use within data logger (1). Thus, data logger (1) can receive and store data from two different kinds of sterilizers: those that output RS-232 serial data, and those that output TTL serial data.

With respect to FIG. 7, RS-232 detector (25) operates on the premise that the idle (mark) state for RS-232 signals from connector socket (21) is a negative voltage in the range of −6V to −12V. This negative voltage keeps two P-channel MOSFETs (33) turned on, which results in keeping an NPN transistor (35) turned off. This results in a logic high (+3.3V) on the 1=16232 output. Bursts of data result in signal transitions to the space state (+6V to +12V), but these are filtered out by an RC network (37) and a diode (39), keeping the two P-channel MOSFETs (33) turned on throughout any data bursts. If the input signals are 5V TTL levels, then the average voltage never goes below ground and the two P-channel MOSFETs (33) remain off. A 10 k resistor (39) turns on NPN transistor (35) resulting in a logic low at the output.

Some versions of RS-232 receiver (27) may comprise a Maxim MAX3180EUK+T. This integrated circuit is a single RS-232 receiver in a SOT23-5 package. When RS-232 signals are present at its input, RS-323 receiver (27) delivers a +3.3V logic level output to multiplexer (31).

Some versions of TTL receiver (29) may comprise a single 74LVC1G17 Schmitt trigger buffer. Its input is protected from positive and negative overvoltage by a diode (43), which may comprise a dual BAT54S Schottky diode with input current limited by a resistor (45), which may comprise a 100 k resistor. In some versions of TTL receiver (29), when the input signal consists of +5V/GND levels, TTL receiver (29) buffers and translates to +3.3V/GND levels. The output feeds multiplexer (31).

Some version of multiplexer (31) may comprise a 2:1 multiplexer. Some versions of multiplexer (31) may comprise a 74LVC1G157 multiplexer. Depending on the input signal it receives from RS-232 detector (25), multiplexer (31) outputs to processor (22) either the RS_DATA signal coming from RS-232 receiver (27) or the TTL_DATA signal coming from TTL receiver (29).

In some versions of serial data input circuit (23), a TVS bidirectional diode (47) protects the entire circuit from overvoltage and transients that exceed ±15V. In some versions of TVS bidirectional diode (47) may comprise a SMAJ15CA bidirectional TVS diode.

ii. Additional Exemplary Circuits

As shown in FIG. 6, data logger (1) may also include an LCD circuit (49). LCD circuit (49) is operably connected to display screen (19) for manipulating the visual output displayed to the user. Some versions of LCD circuit (49) may incorporate an AMC2002CR-B-Y6WFDY-ST-C-S1 LCD module as display screen (19) with a 3.3V parallel interface to processor (22).

Also shown in FIG. 6 is a battery backup and test circuit (51). Processor (22) is configured to accept a battery-powered voltage from battery (53) to provide backup power for a real time clock (RTC) (not shown). Processor (22) utilizes battery (53) to power the RTC when the main power supply is off and the rest of data logger (1) is unpowered. Battery backup and test circuit (51) includes a sub-circuit for testing battery (53), shown as battery test circuit (55).

As shown in FIGS. 5 and 6, data logger (1) is powered by sterilizer (3) via a power connector (57). In some versions of data logger (1), approximately +5V is supplied by sterilizer (3) and fed into a +3.3V 500 mA fixed linear regular (59). Linear regulator (59) in turn provides power to all other circuits within data logger (1), with battery (53) providing backup power for the RTC. In some versions of data logger (1), other components are supplied with the full +5V from a separate incoming feed via a power connector (58). This full +5V may be used to power other components such as display screen (19) and a USB stick, which will be discussed in greater detail below. A manual reset button (61) may be provided along the power pathway in conjunction with a voltage supervisor and reset circuit (63) to allow a user to manually reset data logger (1) by manually actuating the button.

iii. Exemplary Internal Storage Using a MicroSD Card

As shown in FIG. 6, data logger (1) further includes a microSD card holder (65) configured to receive a microSD card (67) to be used as internal storage for data logger (1). MicroSD card holder (65) is operably connected to processor (22) and is configured to receive data from sterilizer (3) via processor (22) and transfer the data to microSD card (67). In operation, the data provided by sterilizer (3) through serial data input circuit (23) is passed to processor (22) and thereafter on to microSD card holder (65) to log the information on microSD card (67). In some versions of data logger (1), microSD card (67) is easily accessible through housing (9) and is selectively removable by the user. Thus, a user can remove microSD card (67) and insert it into another computer system for further processing of the data collected from sterilizer (3) via data logger (1).

While microSD card (67) is shown and described, any similar element for data storage may be used in conjunction with or in place of microSD (67). For example, an SD card, a miniSD card, a memory stick, a “CompactFlash” card, or any other style or format of memory card or data storage element in general.

iv. Exemplary Removable Storage Using a USB Stick

As shown in FIG. 6, data logger (1) further includes a USB connector (69) configured to selectively receive a USB stick (71) to be used as external removable storage for data logger (1). USB connector (69) is operably connected to processor (22) and is configured to receive data from sterilizer (3) via processor (22) and transfer the data to USB stick (71), shown in FIG. 2 as removable storage device (5). In operation, the data provided by sterilizer (3) through serial data input circuit (23) is passed to processor (22) and thereafter on to USB stick (71) to log the information on USB stick (71) if USB stick (71) is present in USB connector (69).

Processor (3) is configured to detect whether USB stick (71) is present and write collected data to USB stick (71) if USB stick (71) is present. In some versions of data logger (1), upon insertion of USB stick (71), processor (22) automatically copies all day from microSD card (67) to USB stick (71). In other versions of data logger (1), upon insertion of USB stick (71), processor (22) actuates a prompt to the user via display screen (19) to query whether the user wishes to have the data stored on microSD card (67) copied to USB stick (71). In other versions of data logger (1), a user must affirmatively interface with display screen (19) to select an option to actuate the copying of data stored on microSD card (67) to USB stick (71).

Similar to microSD card (67), while USB stick (71) is shown and described, any similar element for data storage may be used in conjunction with or in place of USB stick (71). For example, another microSD card, an SD card, a miniSD card, a memory stick, a “CompactFlash” card, or any other style or format of memory card or data storage element in general.

### III. EXEMPLARY DATA LOGGER METHODS

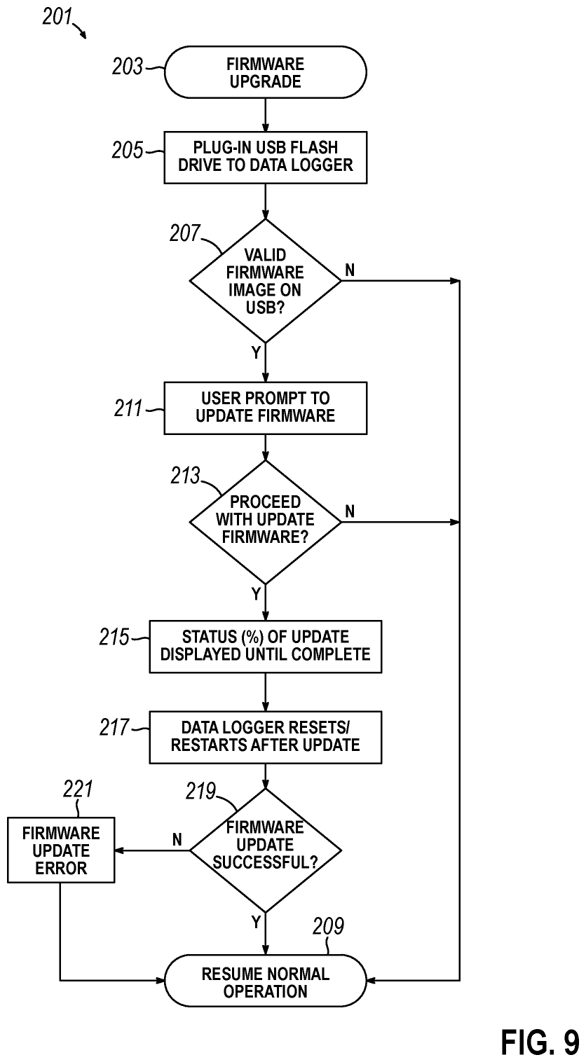

FIG. 8 includes an example of a method (101) for logging data associated with the sterilizer using the data logger. Method (101) begins with a step (103). In step (103), the sterilizer starts a sterilizing cycle, and the method (101) proceeds to step (105). In step (105), the sterilizing cycle is performed, and the method (101) proceeds to step (107). In step (107), the data logger queries whether an error has occurred. If the data logger determines in step (107) that an error has occurred, the data logger proceeds to step (109), wherein the data logger records type “Error” or otherwise formats or includes an error descriptor in the data stream. The error descriptor indicates to the viewer of the record that an error occurred in the sterilizing cycle. If the data logger determines in step (107) that an error has not occurred, then the data logger proceeds to step (111), wherein the data logger queries whether the sterilizing cycle is complete. If the data logger determines in step (111) that the sterilizing cycle is not complete, then the method (101) returns to step (105), wherein the sterilizing cycle is continued to be performed.

If the data logger determines in step (111) that the sterilizing cycle is complete, then the data logger proceeds to step (113). In step (113), the data logger queries whether to record an acceptance or rejection of the sterilizing cycle (e.g., by requesting user input). If the data logger determines an acceptance in step (113), then the data logger proceeds to step (115), wherein the data logger queries whether a signoff user has been selected. If the data logger determines in step (115) that a signoff user has been selected, then the data logger proceeds to step (117), wherein the data logger records type “Accepted” or otherwise formats or includes an acceptance descriptor in the data stream. The acceptance descriptor indicates to the viewer of the record that the record of the sterilizing cycle was accepted by the user after the sterilizing cycle. The signoff user may also be included in the data stream. Alternatively, step (115) may prompt the user to enter a personal identifier and thereafter include the personal identifier in the data stream. If the data logger determines in step (115) that a signoff user has not been selected or a personal identifier is not entered, then the data logger proceeds to step (119), wherein the data logger records type “Blank.”

If the data logger determines a rejection in step (113), then the data logger proceeds to step (121). In step (121), the data logger queries whether a reason for the rejection was selected by the operator. If the data logger determines in step (121) that a reason was selected, then the data logger proceeds to step (123), wherein the data logger records type “Rejected.” If the data logger determines in step (121) that a reason was not selected, then the data logger proceeds to step (119), wherein the data logger records type “Blank.”

If the data logger determines neither an acceptance nor a rejection in step (113), then the data logger proceeds to step (119), wherein the data logger records type “Blank.”

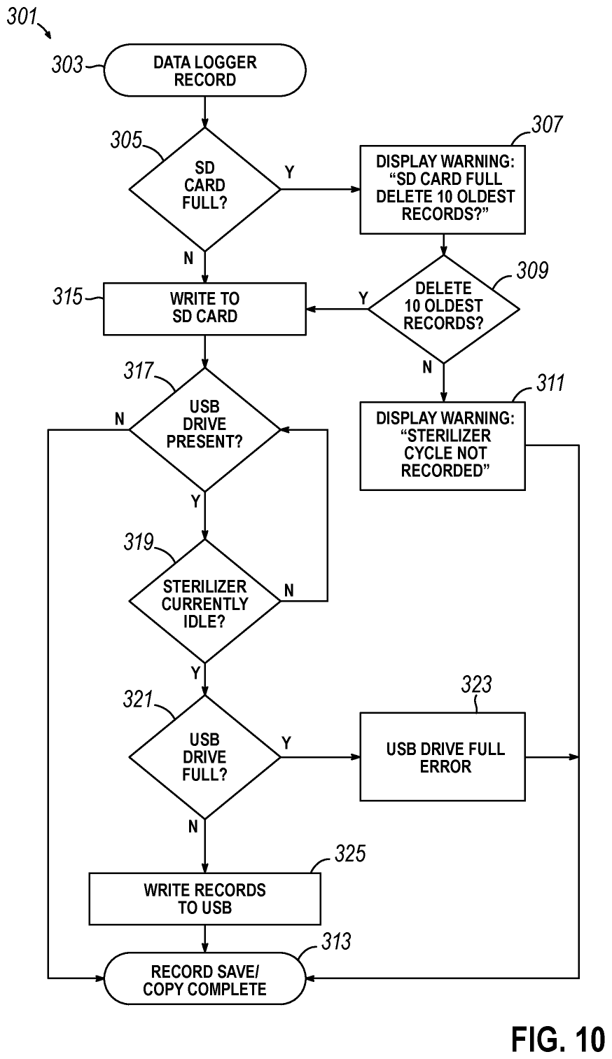

FIG. 9 includes an example of a method (201) for updating the firmware of a data logger similar to data logger (1). Method (201) begins with a step (203). In step (203), a firmware upgrade is desired. The method (201) proceeds from step (203) to step (205). In step (205), a user plugs a USB flash drive (or other removable storage device) into the data logger, and the method (201) proceeds from step (205) to step (207). In step (207), the data logger queries whether a valid firmware image is on the USB flash drive. If the data logger determines in step (207) that there is not a valid firmware image on the USB flash drive, then the data logger proceeds to step (209), wherein the data logger resumes normal operation.

If the data logger determines in step (207) that there is a valid firmware image on the USB flash drive, then the data logger proceeds to step (211). In step (211), the user is prompted to update the firmware of the data logger, and step (211) proceeds to step (213). In step (213), the data logger queries whether to proceed with updating the firmware of the data logger (e.g., by requesting user input). If the data logger determines in step (213) not to proceed with updating the firmware of the data logger, then the data logger proceeds to step (209), wherein the data logger resumes normal operation.

If the data logger determines in step (213) to proceed with updating the firmware of the data logger, then the data logger proceeds to step (215). In step (215), the status of the firmware update (e.g., percentage of completion) is displayed until the firmware update is complete. The data logger then proceeds from step (215) to step (217). In step (217), the data logger resets and/or restarts after the firmware update is complete. The data logger then proceeds from step (217) to step (219). In step (219), the data logger queries whether the firmware update was successful. If the data logger determines in step (219) that the firmware update was successful, then the data logger proceeds to step (209), wherein the data logger resumes normal operation.

If the data logger determines in step (219) that the firmware update was not successful, then the data logger proceeds to step (221). In step (221), the data logger provides a firmware update error which may include, for example, a visual and/or audible alert for communicating the error to the user. The data logger then proceeds from step (221) to step (209), wherein the data logger resumes normal operation.

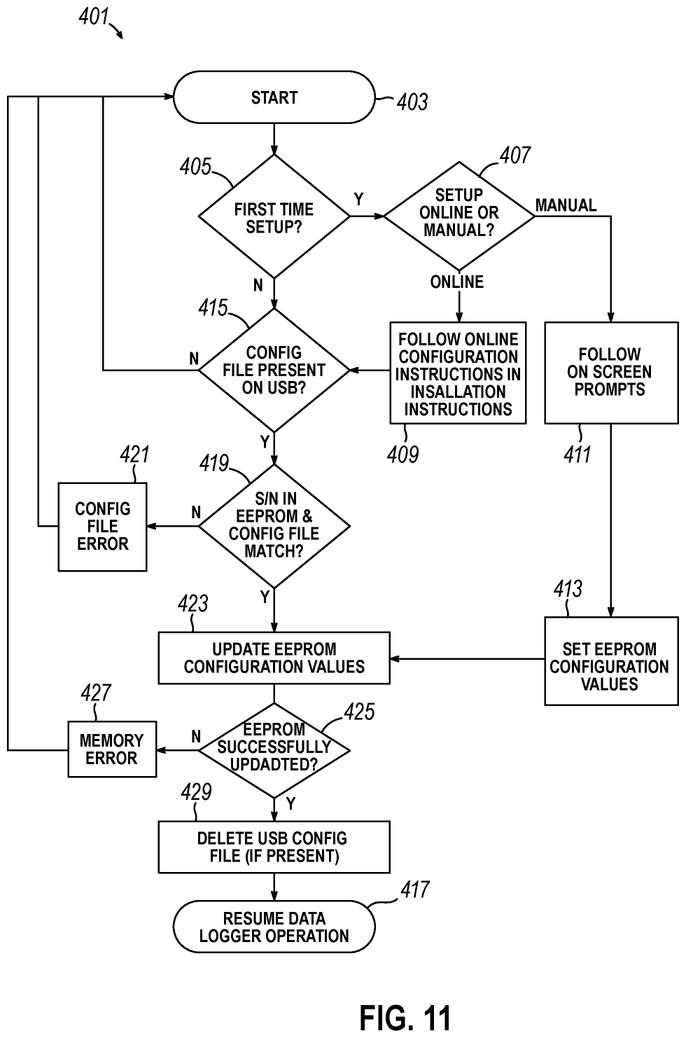

FIG. 10 includes an example of a method (301) for saving and/or copying one or more records of a data logger similar to data logger (1) and associated with a cycle of a sterilizer similar to sterilizer (3). Method (301) begins with a step (303). In step (303), at least one data logger record associated with a cycle of the sterilizer is provided, and the method (301) proceeds from step (303) to step (305). In step (305), the data logger queries whether the SD card is full, such as by querying whether the SD card has insufficient available storage space to store the current record. If the data logger determines in step (305) that the SD card is full, then the data logger proceeds to step (307), wherein the data logger displays a warning indicating that the SD card is full and requesting user input regarding whether to delete records from the SD card (e.g., the 10 oldest records) to free up storage space on the SD card. The data logger proceeds from step (307) to step (309), wherein the data logger queries whether to delete records from the SD card (e.g., the 10 oldest records), such as based on the user input requested in step (307). If the data logger determines in step (309) not to delete records from the SD card, then the data logger proceeds to step (311). In step (311), the data logger displays a warning indicating that the sterilizer cycle has not been recorded, and the data logger proceeds from step (311) to step (313), wherein the record saving/copying process is complete.

If the data logger determines in step (305) that the SD card is not full, or if the data logger determines in step (309) to delete records from the SD card to free up storage space on the SD card, then the data logger proceeds to step (315). In step (315), the data logger writes the one or more records associated with the cycle of the sterilizer to the SD card. The data logger then proceeds from step (315) to step (317).

In step (317), the data logger queries whether a USB flash drive is present. If the data logger determines in step (317) that a USB flash drive is not present, then the data logger proceeds to step (313), wherein the record saving/copying process is complete.

If the data logger determines in step (317) that a USB flash drive is present, then the data logger proceeds to step (319). In step (319), the data logger queries whether the sterilizer is currently idle. If the data logger determines in step (319) that the sterilizer is not currently idle, then the data logger returns to step (317), wherein the data logger again queries whether a USB flash drive is present. If the data logger determines in step (319) that the sterilizer is currently idle, then the data logger proceeds to step (321). In step (321), the data logger queries whether the USB flash drive is full. If the data logger determines in step (321) that the USB flash drive is full, then the data logger proceeds to step (323). In step (323), the data logger provides a USB flash drive full error which may include, for example, a visual and/or audible alert for communicating the error to the user, and the data logger proceeds from step (323) to step (313), wherein the record saving/copying process is complete.

If the data logger determines in step (321) that the USB flash drive is not full, then the data logger proceeds to step (325). In step (325), the data logger writes the one or more records associated with the cycle of the sterilizer to the USB flash drive. The data logger then proceeds from step (325) to step (313), wherein the record saving/copying process is complete.

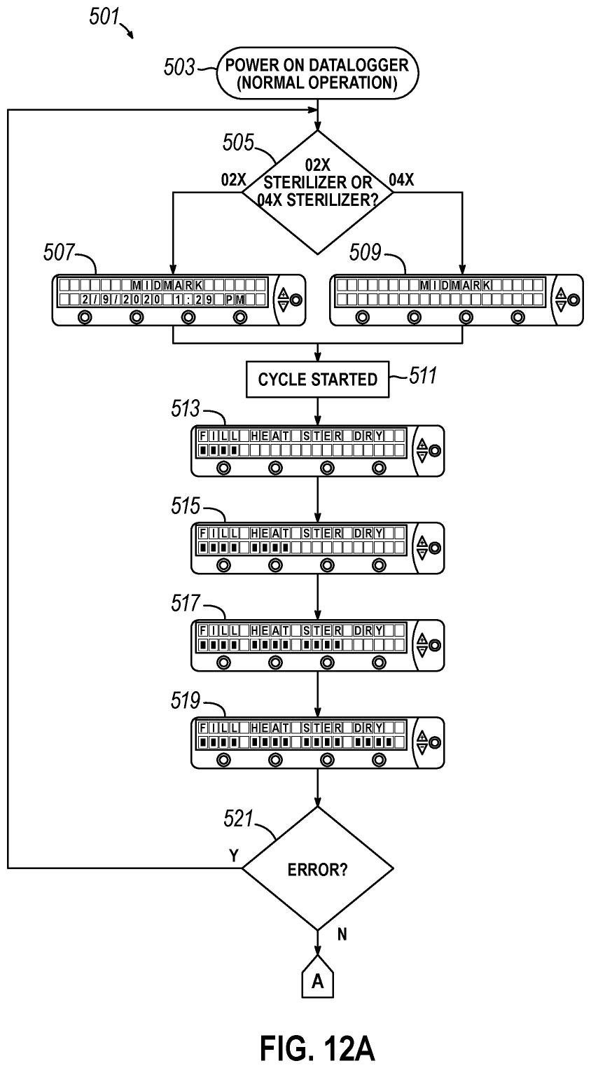

FIG. 11 includes an example of a method (401) for setting up a data logger similar to data logger (1). Method (401) begins with a step (403). In step (403), the data logger starts up and proceeds to step (405). In step (405), the data logger queries whether the present startup is the first time that the data logger has undergone a setup procedure. If the data logger determines in step (405) that the present startup is the first time that the data logger has undergone a setup procedure (i.e., that the data logger has not previously been through the first time setup process), then the data logger proceeds to step (407). In step (407), the data logger queries whether to perform the setup procedure online or manually (e.g., by requesting user input). If the data logger determines in step (407) to perform the setup procedure online, then the data logger proceeds to step (409), wherein the user is instructed to follow the online configuration instructions in the installation instructions for completing the setup procedure. If the data logger determines in step (407) to perform the setup procedure manually, then the data logger proceeds to step (411), wherein the user is instructed to follow on-screen prompts for completing the setup procedure. The data logger proceeds from step (411) to step (413), wherein the data logger sets the EEPROM configuration values.

If the data logger determines in step (405) that the present startup is not the first time that the data logger has undergone a setup procedure (i.e., that the data logger has previously been through the first time setup process), then the data logger proceeds to step (415). Alternatively, the data logger may proceed from step (409) to step (415). In step (415), the data logger queries whether a configuration file is present on the USB flash drive. If the data logger determines in step (415) that a configuration file is not present on the USB flash drive, then the data logger proceeds back to step (403), wherein the user resumes the first-time setup by restarting method (401).

If the data logger determines in step (415) that a configuration file is present on the USB flash drive, then the data logger proceeds to step (419). In step (419), the data logger queries whether the serial number in EEPROM the serial number in the configuration file match. If the data logger determines in step (419) that the serial number in EEPROM and the serial number in the configuration file do not match, then the data logger proceeds to step (421). In step (421), the data logger provides a configuration file error which may include, for example, a visual and/or audible alert for communicating the error to the user. The data logger then proceeds from step (421) to step (403), wherein the user resumes the first-time setup by restarting method (401).

If the data logger determines in step (419) that the serial number in EEPROM and the serial number in the configuration file match, then the data logger proceeds to step (423). Alternatively, the data logger may proceed from step (413) to step (423). In step (423), the data logger updates the EEPROM configuration values. The data logger proceeds from step (423) to step (425). In step (425), the data logger queries whether the EEPROM was successfully updated. If the data logger determines in step (425) that the EEPROM was not successfully updated, then the data logger proceeds to step (427). In step (427), the data logger provides a memory error which may include, for example, a visual and/or audible alert for communicating the error to the user. The data logger then proceeds from step (427) to step (403), wherein the user resumes the first-time setup by restarting method (401).

If the data logger determines in step (425) that the EEPROM was successfully updated, then the data logger proceeds to step (429). In step (429), the data logger deletes the configuration file (if present) from the USB flash drive. The data logger then proceeds from step (429) to step (417), wherein the data logger resumes data logger operation.

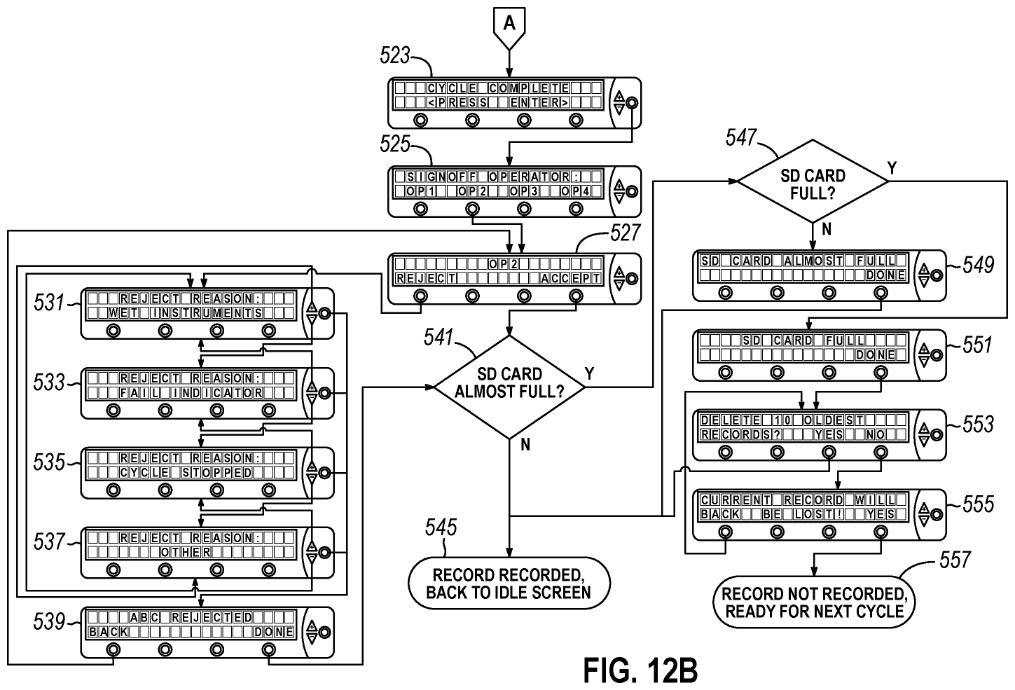

FIGS. 12A and 12B include an example of a method (501) for interfacing a data logger similar to data logger (1) with a user. Method (501) begins with a step (503). In step (503), the data logger is powered on for normal operation, and proceeds to step (505). In step (505), the data logger queries whether the data logger is connected to a 02× sterilizer board or to a 04× sterilizer board. If the data logger determines in step (505) that the data logger is connected to a 02× sterilizer board, then the data logger proceeds to step (507), wherein the data logger displays an idle screen including the sterilizer name and the current date and time. If the data logger determines in step (505) that the data logger is connected to a 04× sterilizer board, then the data logger proceeds to step (509), wherein the data logger displays an idle screen including the sterilizer name.

The data logger proceeds from step (507) or step (509) to step (511), wherein a sterilizing cycle is started. The data logger then proceeds from step (511) to steps (513)-(519), wherein the data logger displays various progress screens indicating the current degree of completion of the sterilizing cycle. For example, in step (513), the data logger displays a progress screen indicating completion of the fill portion of the sterilizing cycle. The data logger proceeds from step (513) to step (515), wherein the data logger displays a progress screen indicating completion of the fill and heat portions of the sterilizing cycle. The data logger then proceeds from step (515) to step (517), wherein the data logger displays a progress screen indicating completion of the fill, heat, and sterilize portions of the sterilizing cycle. The data logger proceeds from step (517) to step (519), wherein the data logger displays a progress screen indicating completion of the fill, heat, sterilize, and dry portions of the sterilizing cycle.

The data logger proceeds from step (519) to step (521). In step (521), the data logger queries whether an error has occurred. If the data logger determines in step (521) that an error has occurred, then the data logger returns to step (505) and displays an idle screen in accordance with either step (507) or step (509). If the data logger determines in step (521) that an error has not occurred, then the data logger proceeds to step (523). In step (523), the data logger displays a completion screen indicating that the sterilizing cycle is complete and requesting user input to continue, such as by prompting the user to press the “Enter” button of the data logger. In response to receiving such user input, the data logger proceeds from step (523) to step (525).

In step (525), the data logger displays an operator signoff screen requesting user input to continue, such as by prompting the user to select one of a variety of displayed operator identifiers corresponding to the action buttons of the data logger. For example, the user may select one of “Op1,” “Op2,” “Op3,” or “Op4” by pressing the corresponding action button. In response to receiving such user input in step (525), the data logger proceeds to step (527). In step (527), the data logger displays an approval screen indicating the selected operator identifier (e.g., “Op2”) and requesting user input to continue, such as by prompting the user to indicate an approval status of the sterilizing cycle via the action buttons of the data logger. For example, the user may select one of “Reject” for rejecting the sterilizing cycle by pressing the corresponding action button or “Accept” for accepting the sterilizer cycle by pressing the corresponding action button.

In response to receiving user input in step (527) indicating that the sterilizing cycle was rejected, the data logger proceeds to steps (531)-(537), wherein the data logger displays various rejection reason screens requesting user input to continue, such as by prompting the user to select the applicable reason for rejecting the sterilizing cycle. For example, in step (531), the data logger displays a rejection reason screen indicating a first potential reason for rejecting the sterilizing cycle due to “Wet Instruments.” In step (533), the data logger displays a rejection reason screen indicating a second potential reason for rejecting the sterilizing cycle due to “Fail Indicator.” In step (535), the data logger displays a rejection reason screen indicating a third potential reason for rejecting the sterilizing cycle due to “Cycle Stopped.” In step (537), the data logger displays a rejection reason screen indicating a fourth potential reason for rejecting the sterilizing cycle due to “Other.” The user may scroll between the various rejection reason screens in accordance with steps (531)-(537) until the applicable reason for rejecting the sterilizing cycle is displayed, at which point the user may select the applicable reason by pressing the “Enter” button of the data logger. In response to receiving user input in any of steps (531)-(537) indicating the applicable reason for rejecting the sterilizing cycle, the data logger proceeds to step (539).

In step (539), the data logger displays a confirmation screen indicating that the sterilizing cycle has been rejected by “ABC”—which is an identifier of the person rejecting the sterilizing cycle. In this case, “ABC” reflects a person's initials. The confirmation screen also requests user input to continue, such as by prompting the user to indicate whether to proceed or return to a previous step via the action buttons of the data logger. For example, the user may select “Back” for returning to a previous step by pressing the corresponding action button and the data logger may, in response to receiving such user input, return to step (527) wherein the data logger may again prompt the user to indicate an approval status of the sterilizing cycle via the action buttons of the data logger. Alternatively, the user may select “Done” for proceeding by pressing the corresponding action button.

In response to receiving user input in step (527) indicating that the sterilizing cycle was accepted, or in response to receiving user input in step (539) to proceed, the data logger proceeds to step (541). In step (541), the data logger queries whether the SD card is (at least) almost full, such as by querying whether the SD card has exceeded a predetermined threshold of used storage space.

If the data logger determines in step (541) that the SD card is not almost full, then the data logger proceeds to step (543). In step (543), the data logger displays a confirmation screen indicating the selected operator identifier (e.g., “Op2”) and the approval status of the sterilizing cycle (e.g., “Accepted”) and requesting user input to continue, such as by prompting the user to indicate whether to proceed or return to a previous step via the action buttons of the data logger. For example, the user may select “Back” for returning to a previous step by pressing the corresponding action button and the data logger may, in response to receiving such user input, return to step (527) wherein the data logger may again prompt the user to indicate an approval status of the sterilizing cycle via the action buttons of the data logger. Alternatively, the user may select “Done” for proceeding by pressing the corresponding action button and the data logger may, in response to receiving such user input, proceed to step (545) wherein the data logger records the record. In one embodiment, the data logger may then return to step (505) and display an idle screen in accordance with either step (507) or step (509).

If the data logger determines in step (541) that the SD card is (at least) almost full, then the data logger proceeds to step (547). In step (547), the data logger queries whether the SD card is full, such as by querying whether the SD card has insufficient available storage space to store the current record. If the data logger determines in step (547) that the SD card is not full, then the data logger proceeds to step (549). In step (549), the data logger displays a warning screen indicating that the SD card is almost full and requesting user input to continue, such as by prompting the user to indicate to proceed via the action buttons of the data logger. For example, the user may select “Done” for proceeding by pressing the corresponding action button and the data logger may, in response to receiving such user input, proceed to step (545) wherein the data logger records the record. In one embodiment, the data logger may then return to step (505) and display an idle screen in accordance with either step (507) or step (509).

If the data logger determines in step (547) that the SD card is full, then the data logger proceeds to step (551). In step (551), the data logger displays a warning screen indicating that the SD card is full and requesting user input to continue, such as by prompting the user to indicate to proceed via the action buttons of the data logger. For example, the user may select “Done” for proceeding by pressing the corresponding action button and the data logger may, in response to receiving such user input, proceed to step (553). In step (553), the data logger displays a record deletion authorization screen requesting user input to continue, such as by prompting the user to indicate whether authorization is granted to delete records from the SD card (e.g., the 10 oldest records) to free up storage space on the SD card. For example, the user may select “Yes” for authorizing the deletion of records to free up storage space on the SD card by pressing the corresponding action button and the data logger may, in response to receiving such user input, proceed to step (545) wherein the data logger records the record (e.g., following the authorized deletion of the 10 oldest records). In one embodiment, the data logger may then return to step (505) and display an idle screen in accordance with either step (507) or step (509). Alternatively, the user may select “No” for prohibiting the deletion of records to free up storage space on the SD card by pressing the corresponding action button and the data logger may, in response to receiving such user input, proceed to step (555).

In step (555), the data logger displays a confirmation screen indicating that the current record will be lost and requesting user input to continue, such as by prompting the user to indicate whether to proceed or return to a previous step via the action buttons of the data logger. For example, the user may select “Back” for returning to a previous step by pressing the corresponding action button and the data logger may, in response to receiving such user input, return to step (553) wherein the data logger may again prompt the user to indicate whether authorization is granted to delete records from the SD card to free up storage space on the SD card. Alternatively, the user may select “Yes” for proceeding by pressing the corresponding action button and the data logger may, in response to receiving such user input, proceed to step (557) wherein the data logger does not record the record and is ready for the next sterilization cycle. In one embodiment, the data logger may then return to step (505) and display an idle screen in accordance with either step (507) or step (509).

### IV. EXEMPLARY COMBINATIONS

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A method comprising: (a) connecting a data logger to a sterilizer, wherein the data logger is configured to receive and process incoming data from the sterilizer formatted in either a first data transfer protocol or a second data transfer protocol; (b) performing a sterilizing cycle by the sterilizer; and (c) after the sterilizing cycle, transmitting a data stream from the sterilizer to the data logger, wherein the data stream is formatted in the first data transfer protocol.

### Example 2

The method of the previous or subsequent Examples, further comprising storing at least a portion of the data stream on a first memory device connected to a first memory holder of the data logger.

### Example 3

The method of any of the previous or subsequent Examples, wherein the first memory device comprises a microSD card.

### Example 4

The method of any of the previous or subsequent Examples, further comprising: (a) detecting whether a second memory device is connected to a second memory holder of the data logger; and (b) upon determining a second memory device is connected to the second memory holder, transferring at least a portion of the data stream stored on the first memory device to the second memory device.

### Example 5

The method of any of the previous or subsequent Examples, wherein the second memory device comprises a USB stick.

### Example 6

The method of any of the previous or subsequent Examples, further comprising: (a) disconnecting the data logger from the sterilizer; (b) connecting the data logger to a second sterilizer; (c) performing a second sterilizing cycle by the second sterilizer; and (d) after the second sterilizing cycle, transmitting a second data stream from the second sterilizer to the data logger, wherein the second data stream is formatted in the second data transfer protocol.

### Example 7

The method of any of the previous or subsequent Examples, further comprising: (a) prior to transmitting the data stream from the sterilizer to the data logger, determining whether an error has occurred in the sterilizing cycle; and (b) in response to determining the error has occurred in the sterilizing cycle, including an error descriptor in the data stream.

### Example 8

The method of any of the previous or subsequent Examples, further comprising: (a) in response to determining the error has not occurred in the sterilizing cycle, prompting a user to accept or reject a record of the sterilizing cycle; and (b) in response to the user accepting the record of the sterilizing cycle, including an acceptance descriptor in the data stream.

### Example 9

The method of any of the previous or subsequent Examples, further comprising: (a) in response to the user accepting the record of the sterilizing cycle, prompting the user to enter a personal identifier; and (b) including the personal identifier in the data stream.

### Example 10

The method of any of the previous or subsequent Examples, further comprising: (a) in response to the user accepting the record of the sterilizing cycle, prompting the user to select a signoff user; and (b) including the selected signoff user in the data stream.

### Example 11

The method of any of the previous or subsequent Examples, further comprising inserting the data logger into a slot defined by the sterilizer.

### Example 12

A method comprising: (a) connecting a data logger to a first sterilizer; (b) performing a sterilizing cycle by the first sterilizer; (c) after the sterilizing cycle, transmitting a first data stream from the first sterilizer to the data logger, wherein the first data stream is formatted in a TTL protocol for serial communication; (d) disconnecting the data logger from the first sterilizer; (e) connecting the data logger to a second sterilizer; (f) performing a second sterilizing cycle by the second sterilizer; and (g) after the second sterilizing cycle, transmitting a second data stream from the second sterilizer to the data logger, wherein the second data stream is formatted in a RS-232 protocol for serial communication.

### Example 13

The method of any of the previous or subsequent Examples, further comprising storing at least a portion of the first data stream and the second data stream on a first memory device connected to a first memory holder of the data logger.

### Example 14

The method of any of the previous or subsequent Examples, wherein the first memory device comprises a microSD card.

### Example 15

The method of any of the previous or subsequent Examples, further comprising: (a) detecting whether a second memory device is connected to a second memory holder of the data logger; and (b) upon determining a second memory device is connected to the second memory holder, storing at least a portion of the first data stream and the second data stream on the second memory device.

### Example 16

The method of any of the previous or subsequent Examples, wherein the second memory device comprises a USB stick.

### Example 17

The method of any of the previous or subsequent Examples, further comprising: (a) prior to transmitting the data stream from the first sterilizer to the data logger, prompting a user to accept or reject a record of the sterilizing cycle; and (b) in response to the user accepting the record of the sterilizing cycle, including a personal identifier of the user in the first data stream.

### Example 18

A data logger comprising: (a) a serial data input circuit configured to receive a data stream from a sterilizer, wherein the data stream comprises one of a first data transfer protocol and a second data transfer protocol; (b) a first memory holder configured to receive a first memory device; and (c) a processor, wherein the processor is configured to receive data from the serial data input circuit and store the data on the first memory device via the first memory holder.

### Example 19

The data logger of any of the previous or subsequent Examples, further comprising a second memory holder configured to receive a second memory device, wherein the processor is configured to determine whether the second memory device is present in the second memory holder, wherein the processor is configured receive data from the serial data input circuit and store the data on the second memory device via the second memory holder if the processor determines the second memory device is present in the second memory holder.

### Example 20

The data logger of any of the previous Examples, wherein the first data transfer protocol is a TTL protocol for serial communication, wherein the second data transfer protocol is a RS-232 protocol for serial communication.

### V. MISCELLANEOUS

It should be understood that any of the examples described herein may include various other features in addition to or in lieu of those described above. By way of example only, any of the examples described herein may also include one or more of the various features disclosed in any of the various references that are incorporated by reference herein.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Having shown and described various versions of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A method comprising:
(a) connecting a data logger to a sterilizer, wherein the data logger is configured to receive and process incoming data from the sterilizer formatted in either a first data transfer protocol or a second data transfer protocol;
(b) performing a sterilizing cycle by the sterilizer; and
(c) after the sterilizing cycle, transmitting a data stream from the sterilizer to the data logger, wherein the data stream is formatted in the first data transfer protocol.

2. The method of claim 1, further comprising storing at least a portion of the data stream on a first memory device connected to a first memory holder of the data logger.

3. The method of claim 2, wherein the first memory device comprises a microSD card.

4. The method of claim 2 comprising:
(a) detecting whether a second memory device is connected to a second memory holder of the data logger; and
(b) upon determining a second memory device is connected to the second memory holder, transferring at least a portion of the data stream stored on the first memory device to the second memory device.

5. The method of claim 4, wherein the second memory device comprises a USB stick.

6. The method of claim 1, further comprising:
(a) disconnecting the data logger from the sterilizer;
(b) connecting the data logger to a second sterilizer;
(c) performing a second sterilizing cycle by the second sterilizer; and
(d) after the second sterilizing cycle, transmitting a second data stream from the second sterilizer to the data logger, wherein the second data stream is formatted in the second data transfer protocol.

7. The method of claim 1, further comprising:
(a) prior to transmitting the data stream from the sterilizer to the data logger, determining whether an error has occurred in the sterilizing cycle; and
(b) in response to determining the error has occurred in the sterilizing cycle, including an error descriptor in the data stream.

8. The method of claim 7, further comprising:
(a) in response to determining the error has not occurred in the sterilizing cycle, prompting a user to accept or reject a record of the sterilizing cycle; and
(b) in response to the user accepting the record of the sterilizing cycle, including an acceptance descriptor in the data stream.

9. The method of claim 8, further comprising:
(a) in response to the user accepting the record of the sterilizing cycle, prompting the user to enter a personal identifier; and
(b) including the personal identifier in the data stream.

10. The method of claim 1, further comprising inserting the data logger into a slot defined by the sterilizer.

11. The method of claim 1, wherein the first data transfer protocol is a TTL protocol for serial communication, wherein the second data transfer protocol is a RS-232 protocol for serial communication.

12. A method comprising:
(a) connecting a data logger to a first sterilizer;
(b) performing a sterilizing cycle by the first sterilizer;
(c) after the sterilizing cycle, transmitting a first data stream from the first sterilizer to the data logger, wherein the first data stream is formatted in a TTL protocol for serial communication;
(d) disconnecting the data logger from the first sterilizer;
(e) connecting the data logger to a second sterilizer;
(f) performing a second sterilizing cycle by the second sterilizer; and
(g) after the second sterilizing cycle, transmitting a second data stream from the second sterilizer to the data logger, wherein the second data stream is formatted in a RS-232 protocol for serial communication.

13. The method of claim 12, further comprising storing at least a portion of the first data stream and the second data stream on a first memory device connected to a first memory holder of the data logger.

14. The method of claim 13, wherein the first memory device comprises a microSD card.

15. The method of claim 13 comprising:
(a) detecting whether a second memory device is connected to a second memory holder of the data logger; and
(b) upon determining a second memory device is connected to the second memory holder, storing at least a portion of the first data stream and the second data stream on the second memory device.

16. The method of claim 15, wherein the second memory device comprises a USB stick.

17. The method of claim 12, further comprising:
(a) prior to transmitting the data stream from the first sterilizer to the data logger, prompting a user to accept or reject a record of the sterilizing cycle; and
(b) in response to the user accepting the record of the sterilizing cycle, including a personal identifier of the user in the first data stream.

18. A data logger comprising:
(a) a serial data input circuit configured to receive a data stream from a sterilizer, wherein the data stream comprises one of a first data transfer protocol and a second data transfer protocol;
(b) a first memory holder configured to receive a first memory device; and
(c) a processor, wherein the processor is configured to receive data from the serial data input circuit and store the data on the first memory device via the first memory holder.

19. The data logger of claim 18 further comprising a second memory holder configured to receive a second memory device, wherein the processor is configured to determine whether the second memory device is present in the second memory holder, wherein the processor is configured receive data from the serial data input circuit and store the data on the second memory device via the second memory holder if the processor determines the second memory device is present in the second memory holder.

20. The data logger of claim 18, wherein the first data transfer protocol is a TTL protocol for serial communication, wherein the second data transfer protocol is a RS-232 protocol for serial communication.
